(19) United States
(12) Patent Application Publication
(10) Pub. No.: US 2007/0009483 A1
(43) Pub. Date: Jan. 11, 2007

(54) Compositions and methods for the therapeutic treatment of diabetes

(75) Inventors: Ji-Won Yoon, Vernon Hills, IL (US); Chungja Yoon, Rockville, MD (US); Hee-Sook Jeon, Lake Bluff, IL (US); Soungjin Shin, Lake Bluff, IL (US)

Correspondence Address: MCDERMOTT WILL & EMERY LLP;ATTN: INTELLECTUAL PROPERTY DEPTARTMENT, 28 STATE STREET, BOSTON, MA, 02109, US

(73) Assignee: Biotech Institute for International Innovation, Inc.

(21) Appl. No.: 11/347,190

(22) Filed: Feb. 3, 2006

Related U.S. Application Data
(60) Provisional application No. 60/686,649, filed on Jun. 1, 2005.
(60) Provisional application No. 60/671,562, filed on Apr. 15, 2005.
(60) Provisional application No. 60/649,674, filed on Feb. 3, 2005.

(51) Int. Cl. A61K 48/00; C12N 5/08; C12N 15/861

(52) U.S. Cl. 424/93.2; 514/44; 435/456; 435/366

(57) ABSTRACT

The invention provides a vector having a nucleic acid operably linking a promoter, an intron, a secretory leader sequence encoding nucleic acid, a human betacellulin (BTC) encoding nucleic acid, or functional fragment thereof, and a polyadenylation signal sequence, wherein expression of BTC produces a secreted, mature BTC. Also provided is a vector having a nucleic acid operably linking a cytomegalovirus (CMV) promoter and enhancer region, a β-globin chimeric intron, an albumin leader sequence encoding nucleic acid, a human betacellulin (BTC) encoding nucleic acid, or a functional fragment thereof, and an SV40 polyadenylation signal sequence, wherein expression of BTC produces a secreted, mature BTC. A host cell containing the vectors of the invention are also provided. A method of treating or preventing diabetes is further provided. The method includes administering to an individual an effective amount of a viral particle having a vector expressing a secreted, mature human betaculin (BTC) or a functional fragment thereof, the vector comprising a nucleic acid operably linking a promoter, an intron, a secretory leader sequence encoding nucleic acid, a human betacellulin (BTC) encoding nucleic acid, or functional fragment thereof, and a polyadenylation signal sequence.

This application is based on, and claims the benefit of, U.S. Provisional Applications Nos. 60/686,649, filed Jun. 1, 2005, entitled Compositions and Methods for the Therapeutic Treatment of Diabetes; 60/671,562, filed Apr. 15, 2005, entitled Approach for the Cure of Type 1 Diabetes by the Expression of Betacellulin using a Recombinant Vector; and 60/649,674, filed Feb. 3, 2005, entitled Vector Construct. These provisional applications are incorporated herein by reference.

### BACKGROUND OF THE INVENTION

The present invention relates generally to methods for the treatment and prevention of diabetes and, more specifically, to the regeneration and neogenesis of β cells for therapeutic treatment.

In an individual with normal regulation of blood glucose, the pancreatic hormone insulin is secreted in response to increased blood sugar levels. Increased blood glucose generally occurs following a meal and results from insulin action on peripheral tissues such as skeletal muscle and fat. Insulin stimulates cells of these peripheral tissues to actively take up glucose from the blood and convert it to forms for storage. This process is also referred to as glucose disposal. The levels of blood glucose vary from low to normal to high throughout the day within an individual, depending upon whether the person is in the fasting, intermediate, or fed state. These levels are also referred to as hypoglycemia, euglycemia and hyperglycemia, respectively. In the diabetic individual, these changes in glucose homeostasis are disregulated due to either faulty insulin secretion or action, resulting in a chronic state of hyperglycemia.

Diabetes mellitus is a common disorder, with a prevalence of about 4-5%. The risk of developing diabetes increases with increased weight, with as many as 90% of adult onset diabetic patients being obese. Therefore, due to the high incidence of obese adults, the incidence of adult onset diabetes is increasing worldwide. Diabetes mellitus is classified into three major forms. Type 2 diabetes is one form and is also referred to as non-insulin dependent diabetes (NIDDM) or adult-onset diabetes. Type 1 diabetes is the second form and is referred to as insulin-dependent diabetes (IDDM). The third type of diabetes is genetic and is due to mutations in genes controlling pancreatic islet beta (β) cell function. Although the diagnosis of diabetes is based on glucose measurements, accurate classification of all patients is not always possible. Type 2 diabetes is more common among adults and type 1 diabetes dominates among children and teenagers.

Diabetes mellitus of both types 1 and 2 are associated with a shortened life expectancy as well as other complications such as vascular disease and atherosclerosis. Long-term management of diabetes to prevent late complications often includes insulin therapy regardless of whether the patients are classified as type 1 or type 2. Type 1 diabetes is an auto-immune disease which is associated with near complete loss of the insulin producing pancreatic β cells. This loss of β cells results in insulin-dependence for life. Type 1 diabetes can occur at any age and it has been estimated that about 0.3-1% of all newborns in the Caucasian population will develop this disease during their lifetime.

A widely used method of treatment for type 1 diabetes and to some extent type 2 diabetes has classically consisted of insulin maintenance therapy. Such therapy in its simplest form requires the injection of purified or recombinant insulin into a patient following ingestion of a meal or at regular intervals throughout the day to maintain normal blood glucose levels. These injections are required ideally at a frequency of four times per day. Although the above method of treatment provides some benefit to the patient, this method of insulin therapy nevertheless suffers from inadequate blood glucose control as well as requiring a great deal of patient compliance.

Another method of treatment for type 1 diabetes includes the use of devices such as an insulin pump which allows for the scheduled delivery of insulin. This method can be preferable to the method described above due to the need for less frequent injections. However, the use of an insulin pump therapy also has drawbacks in that replacement of a needle once every three days is still required. Similar to insulin maintenance therapy, the insulin pump method also does not achieve optimal glucose regulation as the delivery of insulin is not regulated in response to changes in blood glucose level. These methods of treating diabetes are therefore burdensome as well as inadequate. Furthermore, these methods also have not been completely effective over the course of an average adult lifetime and or have been shown to be effective in preventing this disease.

Various approaches of cell therapy for replacing bioactive insulin into a diabetic individual have been attempted. These include gene therapy approaches, immunotherapies and use of artificial β cells. In vivo gene therapy for the expression of insulin or other polypeptides has included liver targeted viral mediated transduction in animal models. However, these approaches did not provide glucose regulated insulin delivery nor did they restore or regenerate insulin producing β cells and have limited applications in patients.

Genetic modification of pancreatic islet β cells and generation of artificial beta cells are approaches for the treatment of diabetes by cell therapy. Xenograft and even allogeneic cell delivery to express insulin require cell encapsulation to prevent host immune responses, and problems with cell survival and sustained insulin delivery have been identified. Pancreatic and islet transplantation has also been attempted as a treatment for diabetes. Use of this treatment has shown limited success due to the requirement for matched tissue from 2-5 adult donors per recipient. This method has also lacked success due, in part, to the failure of the transplanted tissue to maintain normal glucose-regulated insulin secretion and to remain viable over a reasonable period of time.

Thus, there exists a need for simple and more efficient methods that can regulate glucose homeostasis in a diabetic individual in a way that restores the physiological capacity of insulin producing β cells. The present invention satisfies this need and provides related advantages as well.

### SUMMARY OF THE INVENTION

The invention provides a vector having a nucleic acid operably linking a promoter, an intron, a secretory leader sequence encoding nucleic acid, a human betacellulin (BTC) encoding nucleic acid, or functional fragment thereof, and a polyadenylation signal sequence, wherein expression of BTC produces a secreted, mature BTC. Also provided is a vector having a nucleic acid operably linking a cytomegalovirus (CMV) promoter and enhancer region, a β-globin chimeric intron, an albumin leader sequence encoding nucleic acid, a human betacellulin (BTC) encoding nucleic acid, or a functional fragment thereof, and an SV40 polyadenylation signal sequence, wherein expression of BTC produces a secreted, mature BTC. A host cell containing the vectors of the invention are also provided. A method of treating or preventing diabetes is further provided. The method includes administering to an individual an effective amount of a viral particle having a vector expressing a secreted, mature human betaculin (BTC) or a functional fragment thereof, the vector comprising a nucleic acid operably linking a promoter, an intron, a secretory leader sequence encoding nucleic acid, a human betacellulin (BTC) encoding nucleic acid, or functional fragment thereof, and a polyadenylation signal sequence.

### BRIEF DESCRIPTION OF THE DRAWINGS

FIG. 1 shows the remission of diabetes in STZ-induced diabetic NOD.scid and Balb/c mice by systemic administration of rAd-CMV-BTC.

FIG. 2 shows the remission of diabetes by rAd-CMV-BTC resulted from increased β cell mass and insulin.

FIG. 3 shows that ErbB-2 is involved in the remission of diabetes by rAd-CMV-BTC.

FIG. 4 shows the remission of diabetes in autoimmune diabetic NOD mice by treatment with rAd-CMV-BTC.

### DETAILED DESCRIPTION OF THE INVENTION

This invention is directed to a recombinant vector for in vivo secretion of therapeutic polypeptides ant to methods for treating or preventing diabetes. The vector is particularly useful for expression and secretion of human betacellulin (BTC) for the treatment of diabetes. The methods of the invention are directed to introducing the above recombinant vector encoding a secretable BTC into diabetic individuals for the regeneration or neogenesis of insulin producing pancreatic islet β cells and restoration of glucose homeostasis. An advantage of the vector and its use in treating diabetes is that BTC is secreted into the extracellular environment where it can act through it's normal cell signaling pathways. This in vivo secretion results in the remission of diabetes in patients or its prevention in individuals suspected to be at risk of developing diabetes

In one embodiment, the invention relates to an adenoviral vector expressing human betacellulin (BTC). The adenoviral vector contains the cytomegalovirus (CMV) promoter/enhancer and enhancer region 5′ to the BTC coding region, a β-globin chimeric intron, and an albumin leader sequence, to facilitate secretion of BTC. Located 3′ to the BTC coding region is the SV40 polyadenylation signal sequence. The BTC[1-80] cDNA, encoding mature BTC was inserted between these 5′ and 3′ expression and regulatory regions.

In another embodiment, the invention relates to the treatment of diabetes through administration of a recombinant adenoviral vector containing human BTC cDNA fused with the albumin leader sequence Secreted expression of BTC resulted in complete remission of diabetes within 2 weeks whereas autoimmune diabetic NOD mice treated with immunoregulators remained normoglycemic for over 100 days. Remission of diabetes was due to BTC-mediated regeneration of β cells in the pancreas and was abrogated by inhibition of ErbB-2 receptors, ligands for BTC.

As used herein, the term “diabetes” is intended to mean the diabetic condition known as diabetes mellitus. Diabetes mellitus is a chronic disease characterized by relative or absolute deficiency of insulin which results in glucose intolerance. The term is intended to include all types of diabetes mellitus, including, for example, type I, type II, and genetic diabetes. Type I diabetes is also referred to as insulin dependent diabetes mellitus (IDDM) and also includes, for example, juvenile-onset diabetes mellitus. Type I is primarily due to the destruction of pancreatic β-cells. Type II diabetes mellitus is also known as non-insulin dependent diabetes mellitus (NIDDM) and is characterized, in part, by impaired insulin release following a meal. Insulin resistance can also be a factor leading to the occurrence of type II diabetes mellitus. Genetic diabetes is due to mutations which interfere with the function and regulation of β-cells.

Diabetes is characterized as a fasting level of blood glucose greater than or equal to about 140 mg/dl or as a plasma glucose level greater than or equal to about 200 mg/dl as assessed at about 2 hours following the oral administration of a glucose load of about 75 g. The term “diabetes” is also intended to include those individuals with hyperglycemia, including chronic hyperglycemia and impaired glucose tolerance. Plasma glucose levels in hyperglycemic individuals include, for example, glucose concentrations greater than normal as determined by reliable diagnostic indicators. Such hyperglycemic individuals are at risk or predisposed to developing overt clinical symptoms of diabetes mellitus.

As used herein, the term “treating” is intended to mean an amelioration of a clinical symptom indicative of diabetes. Amelioration of a clinical symptom includes, for example, a decrease in blood glucose levels or an increase in the rate of glucose clearance from the blood in the treated individual compared to pretreatment levels or to an individual with diabetes. The term “treating” also includes an induction of a euglycemic response in the individual suffering from disregulated hyperglycemia. Euglycemia refers to the range of blood glucose levels clinically established as normal, or as above the range of hypoglycemia but below the range of hyperglycemia. Therefore, a euglycemic response refers to the stimulation of glucose uptake to reduce the plasma glucose concentration to normal levels. For most adults, this level corresponds to the range in concentration of about 60-105 mg/dL of blood glucose and preferably between about 70-100 mg/dL, but can vary between individuals depending on, for example, the sex, age, weight, diet and overall health of the individual. Effective treatment of a diabetic individual, for example, would be a reduction in that individual's hyperglycemia, or elevated blood glucose levels, to normalized or euglycemic levels, with this reduction directly resulting from secretion of insulin. Alternatively, effective treatment would be a reduction in fasting blood glucose to levels less than or equal to about 140 mg/dL.

The term “treating” is also intended to include the reduction in severity of a pathological condition or a chronic complication which is associated with diabetes. Such pathological conditions or chronic complications are listed in Table 1 and include, for example, muscle wasting, ketoacidosis, glycosuria, polyuria, polydipsia, diabetic microangiopathy or small vessel disease, atherosclerotic vascular disease or large vessel disease, neuropathy and cataracts.

TABLE 1
Pathological Conditions Associated with Diabetes

```
Kidney
  Glomerular microangiopathy
    Diffuse glomerulosclerosis
    Nodular glomerulosclerosis (Kimmel-stiel-Wilson disease)
  Urinary infections
    Acute pyelonephritis
  Renal Failure
    Necrotizing papillitis
    Emphysematous pyelonephritis
  Glycogen nephrosis (Armanni-Ebstein lesion)
Eye
  Retinopathy
    Nonproliferative retinopathy: capillary Microaneurysms, retinal
    edema exudates, and hemorrhages
    Proliferative retinopathy: proliferation of small vessels
  Visual Failure
    hemorrhage fibrosis, retinal detachment
  Cataracts
  Transient refractive errors due to osmotic changes in lens
  Glaucoma due to proliferation of vessels in the iris
  Infections
Nervous System
  Cerebrovascular atherosclerotic disease: strokes, death
  Peripheral neuropathy; peripheral sensory and motor cranial, autonomic
Skin
  Infections: folliculitis leading to carbuncles
  Necrobiosis lipoidica diabeticorum: due to microangiopathy
  Xanthomas: secondary to hyperlipidemia
Cardiovascular system
  Coronary atherosclerosis: myocardial infarction, death
  Peripheral atherosclerosis: limb ischemia, gangrene
Reproductive system
  Increased fetal death rate (placental disease, neonatal respiratory
  distress syndrome, infection)
General
  Increased susceptibility to infection
  Delayed wound healing
```

Additional complications also include, for example, a general increased susceptibility to infection and wound healing. The term “treating” is also intended to include an increase in the average life expectancy of a diabetic individual compared to a non-treated individual. Other pathological conditions, chronic complications or phenotypic manifestations of the disease are known to those skilled in the art and can similarly be used as a measure of treating diabetes so long as there is a reduction in the severity of the condition, complication or manifestation associated with the disease.

As used herein, the term “preventing” is intended to mean a forestalling of a clinical symptom indicative of diabetes. Such forestalling includes, for example, the maintenance of normal levels of blood glucose in an individual at risk of developing diabetes prior to the development of overt symptoms of the disease or prior to diagnosis of the disease. Therefore, the term “preventing” includes the prophylactic treatment of individuals to guard them from the occurrence of diabetes. Preventing diabetes in an individual is also intended to include inhibiting or arresting the development of the disease. Inhibiting or arresting the development of the disease includes, for example, inhibiting or arresting the occurrence of abnormal glucose metabolism such as the failure to transfer glucose from the plasma into the cells. Therefore, effective prevention of diabetes would include maintenance of glucose homeostasis due to glucose-regulated insulin expression in an individual predisposed to a diabetic condition, for example, an obese individual or an individual with a family history of diabetes. Inhibiting or arresting the development of the disease also includes, for example, inhibiting or arresting the progression of one or more pathological conditions or chronic complications associated with diabetes. Examples of such pathological conditions associated with diabetes are listed in Table 1.

As used herein, the term “betacellulin” or “BTC” is intended to mean a member of the epidermal growth factor family that is expressed in the pancreas and intestine of adult individuals and in the primitive duct cells of the fetal pancreas. The nucleotide and deduced amino acid sequence have been described by, for example, Sasada et al., Biochem Biophys Res Commun. 190:1173-79 (1993) and Shing et al., Science 259:1604-7 (1993). Betacellulin functions to induce regeneration and/or neogenesis of insulin producing β islet cells. A specific example of a nucleotide and amino acid sequence for BTC is set forth below as SEQ ID NOS:1 and 2, respectively, for the human BTC coding region and deduced amino acid sequence. SEQ ID NOS:3-5 provide the amino acid sequences for mature human, bovine and mouse BTC, respectively, while SEQ ID NO:6 provides the consensus amino acid sequence for BTC. The open reading frame of human BTC cDNA encodes a 178-amino acid primary translation product that corresponds to the BTC precursor (pro-BTC). Pro-BTC consists of a number of domains including a presumptive signal peptide (aa¹³⁻²⁶) for localization to the secretory pathway, a short propeptide (aa²⁷⁻³¹), mature BTC containing the EGF motif (aa³²⁻¹¹¹), a short juxtamembrane domain (aa¹¹²⁻¹²⁴), a hydrophobic transmembrane domain (aa¹²⁵⁻¹³⁸) and a cytoplasmic tail domain (aa¹³⁹⁻¹⁷⁸) containing a highly hydrophilic arginine/lysine rich region (aa¹⁴⁶⁻¹⁵⁴).

It is understood that minor modifications can be made without destroying the β cell regeneration or neogenesis activity of the BTC polypeptides or fragments thereof of the invention and that only a portion of the primary structure may be required in order to effect activity. Such modifications are included within the meaning of the term BTC and functional fragment thereof so long as β cell regenerative activity or β cell neogenesis activity is retained. Further, various molecules can be fused to BTC or functional fragments thereof, including for example, other proteins, carbohydrates, lipids or cytotoxic or cytostatic agents. Such modifications are included within the definition of the term.

Minor modifications of peptides having at least about the same β cell regenerative activity or β cell neogenesis activity as a wild type BTC polypeptide include, for example, conservative substitutions of naturally occurring amino acids and as well as structural alterations which incorporate non naturally occurring amino acids, amino acid analogs and functional mimetics. For example, a Lysine (Lys) is considered to be a conservative substitution for the amino acid Arg. Similarly, mimetic structures substituting like charges, such as the positive charged Arg or Lys amino acids, with organic structures having similar charge and spatial arrangements would be considered by those skilled in the art to be a minor modification of a BTC polypeptide, or functional fragment thereof, so long as the resultant BTC polypeptide mimetic exhibits at least about the same β cell regenerative activity or β cell neogenesis activity as the referenced BTC polypeptide.

As used herein, the term “functional fragment” when used in reference to a BTC polypeptide, is intended to mean a portion of BTC which retains at least about the same β cell regenerative activity or β cell neogenesis activity compared to full length BTC. Such functional fragments can include, for example, a derivative of BTC, termed BTC24-76, and having a truncated N-terminal 23 amino acids and C-terminal 4 amino acids. BTC24-76 exhibits 2.5-fold greater activity in differentiation and has one-tenth of the mitogenic activity (Watanabe et al., J. Biol. Chem. 269:9966-73 (1994)).

As used herein, the term “substantially” or “substantially the same” when used in reference to a nucleotide or amino acid sequence of BTC is intended to mean that the nucleotide or amino acid sequence shows a considerable degree, amount or extent of sequence identity when compared to the reference sequence. Such sequence identity is further considered to be significant and meaningful so as to characterize an amino acid sequence or encoding nucleotide sequence as being derived from or related to BTC.

As used herein, the term “vector” refers to a recombinant DNA molecule capable of harboring, propagating or expressing a heterologous nucleic acid. When used in reference to an adeno-associated viral vector, the term is intended to refer to a recombinant DNA molecule having some or all of the DNA of an adeno-associated virus, and also having non-AAV DNA. The non-AAV DNA can encode any desired polypeptide, such as a growth factor, an enzyme, a structural protein, an antibody, or an antigen. The encoded polypeptide can be a full-length polypeptide, or an active or immunogenic fragment of a full-length polypeptide. The non-AAV DNA is placed within the vector such that it is operably linked to an appropriate regulatory element such as a promoter, enhancer or the like.

As used herein, the term “adenoviral vector” refers to a member of the group of parvoviruses characterized by their ability to integrate into a host chromosome in a stable fashion. Adenoviral vectors are well known in the art and can be found described in, for example, Wivel et al., Adenovirus Vectors. Chapter 5 (p. 87-110), and Friedmann T., The Development of Human Gene Therapy. CSHL Press, NY, USA. page 729 (1999). This family of recombination in vivo delivery and expression vectors are capable of infecting a broad range of host cells and tissues and can be easily manipulated to achieve a desired function. A specific example of an adenoviral vector of the invention and an adenoviral vector useful in the therapeutic methods of the invention is described further below in Example I.

Other specific examples of adenoviral vectors include, for example, helper-dependent adenoviral vectors and adeno-associated virus. Helper-dependent adenovectors (gutless adenovirus vectors) have incurred deletion of all viral genes. These adenoviral vectors contain only the cis-acting elements, which encompass the left and right inverted terminal repeat sequences as well as the packaging region needed for encapsulation of the vector genome. Such helper-dependent adenovectors retain about 600 bp of the adenoviral genome. The remaining intervening section is filled with non-coding stuffer DNA. Elimination of the viral DNA ensures that no viral genes are present for expression from the helper-dependent adenovirus backbone. Adeno-associated viral vectors have the advantage of providing a high degree of safety because 96% of the parental adeno-associated viral genome has been deleted. The adeno-associated vectors lack any viral genes, containing instead a recombinant gene of interest.

As used herein, the term “operably linked,” or grammatical equivalents, is intended to mean that the vector components are joined according to well known genetic and cellular principles which allows the requisite function of each component to be carried out on its target nucleic acid. Therefore, an operably linked group of nucleic acid components assembled in a vector are joined in the vector to cause transcription, translation and regulation of the referenced coding region sequence. For example, when operably linked, coding region sequences are fused in frame to ensure translation of the desired full-length polypeptide from the constituent parts.

As used herein, the term “expressing” is intended to mean the transcription and translation of a nucleic acid by a cell. Expression can be, for example, constitutive or regulated such as by an inducible promoter or a tissue or cell specific promoter. Such nucleic acid sequences also can be expressed simultaneously or, alternatively, independently with other desired nucleic acids. Various combinations of these modes of coexpression can additionally be used depending on the number and function of amino acid or nucleotide sequences being expressed. Those skilled in the art know, or can determine, what modes of coexpression can be used to achieve a particular goal or satisfy a desired need. A specific example of constitutive expression using a CMV is described further below in Example I.

As used herein, the term “secretion” or “secreted” is intended to mean expression of a gene product into the extracellular space. Secreted polypeptides employ a leader sequence or signal sequence that directs the propolypeptide to through the cell membrane. In eukaryotic cells, for example, the leader sequence is cleaved off in the rough endoplasmic reticulum to produce a mature polypeptide and the mature polypeptide is trafficked to the cell surface via vesicles. Construction of chimeric gene constructs containing leader sequences operatively linked to a coding region to effect the expression and secretion of mature polypeptide is well known in the art.

As used herein, the term “host cell” refers to a cell to be transformed or transduced by a vector according to the invention. The term also refers to a cell that is capable of being infected by a viral particle containing a vector of the invention as its genome.

As used herein, the term “effective amount” when used in reference to the administration of viral particle having a vector expressing a secreted, mature human betaculin (BTC) or a functional fragment thereof, is intended to mean that the number of administered viral particles is sufficient to infect a target tissue and secrete a therapeutic gene product expressed from the viral particle's genome at a level which will reduce one or more symptoms of diabetes. For example, an effective amount of viral particles secreting BTC consists of the number of particles that would cause a reduction in blood glucose levels or result in glucose homeostasis or both. Moreover, clinical manifestations of diabetes also can be used as a measure of an effective amount of viral particles as described above in Table 1. Similarly, an effective amount of viral particles also is intended to mean the number of viral particles that can be administered and direct the secretion of BTC at sufficient levels to produce a desired effect on a biological or biochemical component cells or tissues of the individual. For example, an effective amount of viral particles secreting BTC also can consist of the number of particles that would cause pancreatic β cell regeneration, β islet cell neogenesis or both β islet cell regeneration and neogenesis. An effective amount of viral particles for a human individual can be, for example, extrapolated from a credible animal model of diabetes given the teachings and guidance provided herein together with that well known by one skilled in the art. An effective amount of viral particles for a mouse animal model, for example, includes between about 1×10⁸-1×10¹⁴, preferably between about 1×10⁹-8×10¹¹, more preferably between about 1×10¹⁰-1×10¹¹. A particularly useful effective amount is about 4×10¹¹ viral particles. Other useful effective amounts include, for example, between about 1×10¹²-1×10¹⁴, particularly when using helper-dependent adenoviral vectors or adeno-associated virus. A particularly useful effective amount for an adeno-associated virus is between about 2.1×10¹²-7.0×10¹³ vector genome units.

As used herein, the term “pharmaceutically acceptable carrier” is intended to mean a solution or media which is appropriate for administration to an individual. Such solutions or media can act to maintain the stability of compounds and polypeptides and the viability of the cells. Pharmaceutically acceptable carriers are well known in the art and include aqueous solutions such as phosphate-buffered saline or media. A pharmaceutically acceptable carrier also includes additional moieties, compounds and/or formulations that act to enhance or increase the ability of the viral particles to target, attach or infect to their in vivo host cells or tissues and/or for timed released delivery or immunoprotection purposes. Such moieties, compounds and/or formulations are well known to those skilled in the art and can include, for example, receptor ligands, extracellular matrix molecules or components thereof and chemical delivery formulations.

Isolated molecules, host cells or populations thereof refer to molecules, host cells or populations which are substantially free of contaminants or material as they are normally found in nature. A population refers to a group of two or more molecules or host cells. Cells which make up a population can be of the same or different lineage and can be a homogenous or heterogenous group of cells.

The invention provides a vector having a nucleic acid operably linking a promoter, an intron, a secretory leader sequence encoding nucleic acid, a human betacellulin (BTC) encoding nucleic acid, or functional fragment thereof, and a polyadenylation signal sequence, wherein expression of BTC produces a secreted, mature BTC.

The invention employs nucleic acids encoding betacellulin together with a secretory leader sequence to produce a propolypeptide, or probetacellulin (pro-BTC), that can be cleaved into a bioactive BTC upon expression through the secretory pathway. Where the BTC is to be employed for human therapeutic purposes, the encoding nucleic acid is preferably from human. Similarly, in such therapeutic uses, the secretory leader sequence also is preferably derived from human sources. Use of encoding nucleic acids for expressed and/or secreted polypeptides, including propolypeptides, reduces the likelihood that the exogenous polypeptide will elicit an unwanted immune response. However, those skilled in the art will know that all other sources of BTC and a secretory leader sequence, including non-human sources, can be utilized in the vector of the invention as well as be employed in the therapeutic methods of the invention, particularly where sequence identity differences with human sequences are small.

An exemplary BTC encoding nucleic acid can have substantially the same nucleotide sequence as the nucleotide sequence set forth as SEQ ID NO:1 for human BTC. Similarly, an exemplary BTC encoding nucleic acid can encode substantially the same amino acid sequence as the amino acid sequence set forth as SEQ ID NO:2 for human BTC. Similarly, an exemplary BTC encoding nucleic acid can encode substantially the same nucleotide sequence as a nucleotide sequence encoding any of the amino acid sequences set forth as SEQ ID NOS:3-6. Minor modifications such as conservative substitutions or differences due to species origin compared to a nucleotide sequence encoding SEQ ID NOS:2-6 also can be employed in the vector of the invention so long as the encoded BTC gene product retains some or all of its β islet regeneration or neogenesis activity.

The encoding nucleic acids of the invention contain sequences corresponding to the coding region of a BTC, or functional fragment thereof, operably linked to a secretory leader sequence. The operable linkage occurs in cis and in such a manner that in vivo cleavage results in the production of a bioactive BTC. The linkage can occur by, for example, direct fusion of the encoding leader sequence to the coding region of BTC or by inclusion of a linker region so long as there is no appreciable diminution of BTC activity following cleavage into the mature polypeptide. The signal peptide cleavage signal can therefore be derived from the chosen leader sequence or from a heterologous leader sequence so long as all of the activities corresponding to secretion, cleavage of the leader sequence and production of active BTC occur. Such nucleic acid sequences encoding BTC and a secretory leader sequence are included in the vector of the invention operably linked with other desired expression and regulatory elements as described below.

A secretory leader sequence can be obtained from essentially any desired eukaryotic polypeptide that is secreted. With the cloning and sequencing of numerous genomes, including human, there exists a wide variety of eukaryotic leader sequences that can be employed. Nucleic acids encoding exemplary leader sequences that can be used in the vector of the invention include, for example, an albumin leader having the sequence ATG AAG TGG GTA ACC TTT ATT TCC CTT CTT TTT CTC TTT AGC TCG GCT TAT TCC AGG GGT GTG TTT CGT CGA GAT (SEQ ID NO:7) and an immunoglobulin kappa (Ig κ)-chain leader having the sequence ATG GAG ACA GAC ACA CTC CTG CTA TGG GTA CTG CTG CTC TGG GTT CCA GGT TCC ACT GGT GAC (SEQ ID NO:8). A particularly useful secretory leader sequence that can be employed in a vector of the invention is the albumin leader sequence as set forth above and exemplified in the Examples.

Any of a variety of expression and regulatory elements can be employed in a vector of the invention to effect the secretion of mature BTC encoded in the vector as described above. Such elements include at least a promoter sequence for transcription of the encoded pro-BTC polypeptide. Other expression elements include, for example, enhancers, silencers, tissue specific transcription regulatory elements, introns, polyadenylation signals, transcription termination signals and translation initiation sites. Where strong, continuous expression is desired, constitutive or inducible promoters used in combination with one or more enhancers can be a particularly useful combination. Similarly, other expression and/or regulatory elements that enhance expression, stability or the transcription, translation or trafficking efficiency also can be employed to beneficially increase the expression and secretion levels of a mature BTC polypeptide of the invention. Such other expression and/or regulatory elements can range from inclusion of structures normally found encoded in a eukaryotic gene such as an intron or polyadenylation signal to substantial modification of a nucleotide sequence to make it more compatible with the codon usage in the target species where expression will occur. For example, a non-human BTC coding region such as rodent can be modified to incorporate some or many human codons in the nucleotide sequence without substantially altering the amino acid sequence. Alternatively, a non-human BTC coding region can be humanized to encode substantially the same human amino acid sequence while differing at the nucleotide level. As described further below, various other combinations and permutations of expression and/or regulatory elements can be employed in the vector of the invention to effect the secretion of a mature BTC polypeptide.

For example, suitable expression and/or regulatory elements are well known to those skilled in the art and are exemplified in, Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1999). Such expression and/or regulatory elements includ the the cytomegalovirus (CMV) promoter, SV40 early promoter, the mouse mammary tumor virus (MMTV) steroid-inducible promoter, Moloney murine leukemia virus (MMLV) promoter, and the like. Expression and/or regulatory elements that provides tissue specific or inducible expression of an operatively linked nucleic acid also can be employed. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.)); metallothionein promoter inducible by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammory tumor virus (MMTV) induced by steroids such as glucocortocoid and estrogen (Lee et al., Nature, 294:228-232 (1981); and heat shock promoters inducible by temperature changes.

A particularly useful promoter for strong constitutive in vivo expression is the CMV promoter and is exemplified further below in Example I. Other particularly useful elements include the operable combination of a CMV promoter and enhancer element, a beta globin introns and a polyadenylation signal sequence. An intron other than a beta globin intron that can be included in a vector of the invention includes, for example, the SV40 large T antigen. Similarly, polyadenylation signals other than SV40 polyadenylation signal that can be included in a vector of the invention include, for example, the bovine growth hormone (BGH) poly A signal and the β-globin poly A signal. All of such elements are commercially available, and their uses are well known in the art. One skilled in the art will know or can readily determine an appropriate promoter for expression in a particular host cell.

The vector of the invention can be derived from a variety of well known sources or produced by a variety of methods well known in the art. For use in therapeutic methods, as described below, the vector is introduced into host cells or tissue so as to effect the expression and secretion of mature BTC from the encoding nucleic acid and the operably linked leader sequence and the operably linked expression and/or regulatory elements. A wide variety of vectors can be employed for this purpose including, expression vectors delivered by targeted gene delivery as well as viral vectors that can be used to produce viral particles for infection of host cells following administration. Viral vectors are particularly useful for in vivo gene delivery because host cell specificity, expression and replication mechanisms, for example, can be beneficially harnessed to achieve efficient introduction and robust expression in a desired cell type or tissue. Both DNA virus-based and retroviral-based vectors can be utilized as a vector of the invention for operable linkage of a promoter, an intron, a secretory leader sequence encoding nucleic acid, a human betacellulin (BTC) encoding nucleic acid, or functional fragment thereof, and a polyadenylation signal sequence, to achieve expression and secretion of mature BTC.

The invention is exemplified by an adenoviral-based vector. The adenoviral component can be derived from, for example, an Ad5 genome or from any other adenoviral-based vectors well known in the art. Another adenoviral-based vector that can be employed as the backbone component of the vector of the invention is a helper-dependent or “gutless” adenoviral vectors (HDAd). This HDAd adenoviral-based vector lacks most of the viral genome and contain only the cis-acting elements (Kochanek et al., Curr. Opin. Mol. Ther. 3:454-463 (2001)). Its use for in vivo delivery of a therapeutic gene produce has shown prolonged expression of the transgene and negligible toxicity (Schiedner et al., Nat. Genet. 18:180-183 (1998)); Zou et al., Mol. Ther. 2:105-113 (2000)), and Morral et al., Hum. Gene Ther. 9:2709-2716 (1998)). In addition, gutless adenovectors have a higher transduction efficiency than, for example, recombinant adeno-associated viruses. The nucleotide sequence for the specific adenoviral vector described below in Example I is set forth as SEQ ID NO:9. The vector contains at nt 1-795 a CMV promoter/enhancer element; at nt 857-989 a β-globin/IgG chimeric introns; at nt 1095-1166 an albumin leader sequence; at nt 1167-1406 a betacellulin[1-80] cDNA coding region; at nt 1407-1409 a stop codon, and at nt 1426-1647 a SV40 late polyA signal. This vector or a vector having substantially the same nucleotide sequence can be employed in the methods of the invention as well.

Further exemplary viral-based vectors include, for example, retroviral, adenovirus, adeno-associated virus, lentivirus, and herpesvirus vectors can be used to express ATX polypeptides into a cell. As described previously, viral based systems provide the advantage of being able to introduce relatively high levels of a heterologous nucleic acid into a variety of cells. Additionally, such viruses can introduce heterologous DNA into nondividing cells. Viral vectors include, for example, Herpes simplex virus vectors (U.S. Pat. No. 5,501,979), Vaccinia virus vectors (U.S. Pat. No. 5,506,138), Cytomegalovirus vectors (U.S. Pat. No. 5,561,063), Modified Moloney murine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, respectively), papilloma virus vectors (U.S. Pat. Nos. 5,674,703 and 5,719,054), and the like.

Methods for construction of a vector of the invention and for the operable linkage of coding sequences and expression and/or regulatory elements are well known in the art. An exemplary expressible nucleic acid sequence encoding BTC containing an albumin secretory leader sequence is provided herein as set forth in SEQ ID NO:10. Methods for constructing a nucleic acid sequence encoding a secretable BTC/albulim pro-BTC are well known in the art, for example, as described by Sambrook et al., supra; Ausubel et al., supra; Kay et al., Hepatology 21:815-819 (1995); Stratford-Perricaudet et al., J. Clin. Invest., 90:626-630 (1992), and Barr et al., Gene Therapy, 2:151-155 (1995). For example, a nucleic acid encoding BTC and containing a secretory leader sequence can be obtained using polymerase chain reaction. A tissue or cell line from the appropriate organism can be used to amplify BTC or leader sequences. Such methods also are exemplified further below in Example I.

Once a vector having a nucleic acid operably linking a promoter, an intron, a secretory leader sequence encoding nucleic acid, a human betacellulin (BTC) encoding nucleic acid, or functional fragment thereof, and a polyadenylation signal sequence has been constructed, the vector can be verified for expression of secreted, mature BTC using methods well known in the art. For example, the vector can be introduced into a cell that does not express BTC and the culture media assayed for the presence of mature insulin using an assay such as ELISA or radioimmunoassay (RIA). Alternatively, the expressed product can be tested for its ability to induce β cell regeneration or β cell neogenesis or decrease the severity of any one of the diabetic symptoms and pathological conditions listed in Table 1. For example, the expressed product can be tested for its ability to stimulate decreases in blood glucose levels or increased transfer of glucose into cultured adipocytes or muscle cells. Measurement of the amount of transfer into the cells can be made by using radiolabelled glucose.

Therefore, the invention also provides a vector having a nucleic acid operably linking a cytomegalovirus (CMV) promoter and enhancer region, a β-globin chimeric interon, an albumin leader sequence encoding nucleic acid, a human betacellulin (BTC) encoding nucleic acid, or a functional fragment thereof, and an SV40 polyadenylation signal sequence, wherein expression of BTC produces a secreted, mature BTC. The nucleotide sequence for the vector can be substantially the same nucleotide sequence as that shown as SEQ ID NO:9.

The invention further provides a host cell containing a BTC-containing vector of the invention. For example, the invention provides a host cell or a population of cells expressing secreted, mature BTC The host cells of the invention can originate from essentially any tissue or organ. For primary cells, a tissue should be selected that is easily accessible and contains cells that exhibit desirable growth and expression characteristics. Additional considerations when selecting a tissue source include choice of a tissue that contains cells that can be isolated, cultured and modified to express BTC in a secreted form. Examples of sources of tissues include pancreas, muscle, liver, or skin tissue, as well as sources of hematopoietic origin. Therefore, cell types within these tissues that can be modified to express secreted, mature BTC can be isolated and employed for purposes including, for example, experimental studies, vector maintenance and passage and for cell therapy protocols. Such cell types include, for example, β islet cells, muscle (smooth, skeletal or cardiac), fibroblast, liver, fat, hematopoietic, epithelial, endothelial, endocrine, exocrine, kidney, bladder, spleen, stem and germ cells. Particularly useful host cells are pancreatic cells, including progenitor and stem cells capable of differentiating into β islet cells. Other cell types are similarly known in the art that are capable of being modified to secrete mature BTC and can similarly be obtained or isolated from a tissue source as described above. Although human tissue sources are advantageous for therapeutic purposes, the species of origin of the cells can be devised from essentially any mammal, so long as the cells exhibit the characteristics that allow for expression and secretion of mature BTC.

The invention also provides a method of treating or preventing diabetes. The method includes administering to an individual an effective amount of a viral particle having a vector expressing a secreted, mature human betaculin (BTC) or a functional fragment thereof, said vector comprising a nucleic acid operably linking a promoter, an intron, a secretory leader sequence encoding nucleic acid, a human betacellulin (BTC) encoding nucleic acid, or functional fragment thereof, and a polyadenylation signal sequence.

In a further embodiment, the vector of the invention can be used to generate viral particles. Administration of such viral particles containing a vector of the invention able to secreted mature BTC following infection can treat and prevent diabetes. For example, a single administration of a recombinant adenoviral vector of the invention containing human BTC fused with the albumin leader sequence resulted in complete remission of diabetes within 2 weeks in streptozotocin-induced diabetic NOD.scid mice and autoimmune diabetic NOD mice treated with immunoregulators, and the mice remained normoglycemic for over 100 days. Remission of diabetes was due to BTC-mediated regeneration of β cells in the pancreas and was abrogated by inhibition of ErbB-2 receptors, ligands for BTC. Regeneration of β cells by BTC gene therapy might be a potential method for the cure of type 1 diabetes in humans.

The viral particle exemplified in the methods of the invention is a adenoviral vector particle. However, as described previously with respect to the vector, given the teachings and guidance provided herein, those skilled in the art will understand that a wide range of viral particles can be employed for stable gene delivery, expression and secretion of mature BTC. Whether adenoviral, other DNA virus-based, retroviral or other, the viral particles harboring a vector of the invention as its genome can be employed in the methods of the invention for the therapeutic treatment or prevention of diabetes as described further below. The viral particles of the invention can be produced, for example, using any of a wide variety of methods well known in the art for packaging viral genomes. Such methods are exemplified below in Example I with respect to an adenoviral particle harboring a vector of the invention.

A diabetic individual lacking glucose homeostasis can be treated with the above-described viral particles by a variety of administration routes and methods. An individual suitable for treatment using the methods of the invention is selected using clinical criteria and prognostic indicators of diabetes that are well known in the art. Definite clinical diagnosis of at least one of the symptoms of diabetes or pathologies related to diabetes as described previously herein would warrant administration of the cells of the invention. A list of exemplary pathological symptoms is included in Table 1.

An individual at risk of developing diabetes as assessed by known prognostic indicators such as family history, fasting blood glucose levels, or decreased glucose tolerance also warrant administration of cells modified to express proinsulin and protease in a glucose-regulated manner. One skilled in the art would recognize or know how to diagnose an individual with diabetes or disregulated glucose uptake and, depending upon the degree or severity of the disease, can make the appropriate determination of when to administer the viral particles of the invention and can also select the most desirable mode of administration. For example, whereas a person with long-standing type 1 disease can require immediate administration of viral particles for infection and secretion of BTC, a person with long-standing type 2 disease could defer treatment until after there is an indication of a lack of effectiveness of other prescribed treatments.

Viral particles having a vector containing a nucleic acid operably linking a promoter, an intron, a secretory leader sequence encoding nucleic acid, a human betacellulin (BTC) encoding nucleic acid, or functional fragment thereof, and a polyadenylation signal sequence that can express a secreted, mature human betaculin (BTC), or a functional fragment thereof, from its vector when introduced into a host cell can be administered to an individual that has been determined to require or benefit from treatment for diabetes for amelioration of their disease. The viral particles can be administered for amelioration of one or more signs or symptoms of diabetes. For example, a diabetic individual can be administered viral particles having a genome coding for secretion mature BTC following diagnosis of the disease. The viral particles will infect target tissues and cells and secrete mature BTC, or a functional fragment thereof, upon in vivo expression of its vector genome. BTC secretion will lead to, for example, β islet cell regeneration in the pancreas, β islet cell neogenesis in the pancreas or both β islet cell regeneration and neogenesis in the pancreas and lead to replenishment of these cells and restoration of glucose homeostasis. An individual that has been effectively treated for diabetes will exhibit a reduction in severity of at least one of the symptoms indicative of the disease following implantation of the insulin secreting cells. The reduction in severity of a symptom can be determined and would be apparent to one skilled in the art.

Individuals with less severe diabetes can also be administered a viral particle of the invention. Determination of a need for treatment in such individuals can be made by one skilled in the art. For example, a diabetic individual that does not respond or responds poorly to standard treatment methods can be treated by methods of the invention. A patient with type 2 disease who has tried unsuccessfully to maintain a long-term decrease in weight or to adhere to an exercise regimen, for example, can be treated for their insulin resistance by implantation of a population of cells of the invention.

The methods of the invention can also be used to improve the efficacy of other therapies for diabetes. The methods of the invention can be used in combination with pre-existing or other methods of treatment to improve the efficacy or ease of use of the other methods. For example, the BTC secreting cells can be produced following administration of the viral particles of the invention in a patient receiving daily injections of insulin or a patient using an insulin pump. Administration of the BTC encoding viral particles and infection with subsequent secretion of BTC can reduce the frequency of insulin injections in such a patient. A diabetic individual not receiving insulin therapy but receiving behavioral modification therapy, for example, diet and exercise to decrease weight, also can be administered the viral particles of the invention. Administration of the BTC viral particles in such individuals, in combination with a weight reduction and exercise regimen, can decrease the likelihood of disease relapse or can ameliorate signs or symptoms of the disease. The BTC encoding viral particles of the invention also can be used to treat a diabetic individual having autoimmune responses against endogenous insulin secreting cells. Such diabetic individuals are often treated by immunotherapeutic intervention of the autoimmune response. These individuals can be additionally treated through the secretion of BTC and regeneration and/or neogenesis of β islet cells to achieve greater therapeutic efficacy than would be achieved with immunotherapy alone.

The viral particles of the invention, which introduce and express a secreted, mature BTC, can be administered to the individual to produce an increase in β islet cell function and thereby insulin secretion to restore or augment glucose-uptake response. Integration of the viral particle genome allows prolonged glucose homeostasis due to the expression restoration of these functions. An individual suffering from diabetes can be administered an effective amount of viral particles to reduce or prevent diabetes. Such an individual could have a fasting blood glucose level of about 140 mg/dl or greater.

An effective amount of viral particles suitable for implantation consists of a size or particle number that is within a range that can be obtained, modified to operably encode secreted, mature BTC and is sufficient to express quantities of secreted, mature BTC, or a functional fragment thereof, following infection of the virus into a target cell or tissue that is therapeutically beneficial in vivo. An effective amount of viral particles for a human individual can be, for example, extrapolated from a credible animal model of diabetes given the teachings and guidance provided herein together with that well known by one skilled in the art. An effective of viral particles for a mouse animal model, for example, includes between about 1×10⁸-1×10¹², preferably between about 1×10⁹-8×10¹¹, more preferably between about 1×10¹⁰-1×10¹¹. A particularly useful effective amount is about 4×10¹¹ viral particles. Choice of virus particle number can depend on the source of the particles, condition of the recipient individual, and the level of BTC secretion required. One skilled in the art will know, using methods well known in the art, how to determine the appropriate number of viral particles that produce a therapeutic effect.

Administration of the viral particles of the invention for delivery of nucleic acids encoding secretable BTC can be by a variety of routes. In addition to intravenously injection (i.p.), an effective amount of viral particles also can be administered into an individual by, for example, injection intramuscularly, subcutaneously, intraperitoneally, or into a tissue or organ site. Viral particles used for administration are obtained and prepared by methods well known in the art and suspended in an appropriate physiological carrier. For example, the viral particles can be infused either directly through a catheter connected to a device containing the particles and the catheter inserted into a vein, or can be injected directly into a tissue. The viral particles are injected in a pharmaceutically acceptable carrier which is defined above and further discussed below. The viral particles also can be administered with other molecules which facilitate delivery, targeting and/or therapeutic efficacy. The viral particles can be administered in single or multiple administrations as necessary to achieve sufficient expression of therapeutic levels of secreted, mature BTC, or a functional fragment thereof.

The individual treated with the viral particles can then be monitored for efficacy of the treatment by measurement of levels of insulin secretion following ingestion of a meal. This measurement can consist of radioimmunoassay measurement or ELISA of, for example, insulin blood levels. Alternatively, measurement of fasting blood glucose levels in the individual following administration of the viral particles can be used to determine efficacy of the treatment. A decreased rate of glucose disposal as determined by a glucose tolerance test also can be used to verify efficacy of the treatment. Additionally, the alleviation of at least one of the symptoms associated with diabetes can also be used to determine efficacy of the treatment. One skilled in the art would know the appropriate means of evaluating and diagnosing efficacy of the treatment.

The invention can also be used for the prevention of diabetes. For example, viral particles encoding secretable BTC can be administered as a prophylactic to an individuals at risk of developing diabetes or suffering from hyperglycemia. The invention can also be used, for example, in individuals genetically predisposed to developing diabetes or in obese individuals at risk for developing insulin resistance or disregulated hyperglycemia. These individuals can receive an effective amount of BTC encoding viral particles for infection of target cells and subsequent secretion of mature BTC prior to or during the onset of clinically overt hyperglycemia. The latter case can be considered as preventing the disease but can also be considered as treating the disease because normal glucose homeostasis is obtained before chronic elevated blood glucose levels are indicated.

In addition to administering BTC encoding viral particles for infection and secretion of BTC in an individual, the vectors of the invention also can be directly administered to an individual for genetic modification, for example, for ex vivo and in vivo therapy.

The viral particles or vectors of the invention containing a nucleic acid secretable BTC can be introduced directly into an individual or formulated as a pharmaceutical composition including a pharmaceutically acceptable carrier. Pharmaceutically acceptable carriers are well known in the art and include aqueous solutions such as water, physiologically buffered saline, or other solvents or vehicles such as glycols, glycerol, oils such as olive oil or injectable organic esters.

A pharmaceutically acceptable carrier can contain physiologically acceptable compounds that act for example, to stabilize or increase the infection of the viral particle, absorption of the vector nucleic acid sequence or both. One skilled in the art will know that the choice of a pharmaceutically acceptable carrier, including a physiologically acceptable compound, depends, for example, on the route of administration of the BTC encoding viral particles and on the particular characteristics of the viral particles, for example, whether the viral particles are based on DNA viruses or retroviruses.

The pharmaceutical composition also can be incorporated, if desired, into oil-in-water emulsions, microemulsions, micelles, mixed micelles, liposomes, microspheres or other polymer matrices (Gregoriadis, Liposome Technology, Vols. I to III, 2nd ed., CRC Press, Boca Raton, Fla. (1993); Fraley et al., Trends Biochem Sci., 6:77 (1981). Liposomes, for example, which consist of phospholipids or other lipids, are nontoxic, physiologically acceptable and metabolizable carriers that are relatively simple to make and administer. In addition, liposomes are particularly useful because they can encapsulate the BTC encoding vectors of the invention high efficiency while not compromising the biological activity of the agent, preferentially and substantially bind to a target cell, and deliver the aqueous contents of the vesicle into the target cell with high efficiency (see Mannino et al., Biotechniques 6:682 (1988)).

Targeting of a liposome for delivery of a vector of the invention to an individual can be passive or active. Passive targeting, for example, uses the tendency of liposomes to accumulate in cells of the reticuloendothelial system (RES) and in an organ such as the liver, which contains sinusoidal capillaries. The vectors formulated as liposomes can be infused directly into the portal vein of the liver and will effectively modify liver cells to express insulin due to the concentration of RES cells in the liver and the sinusoidal nature of the circulatory system in the liver. Active targeting of liposomes containing a vector can be achieved by coupling a specific ligand to the liposome. Such ligands include a monoclonal antibody, a sugar, a glycolipid or a protein such as a ligand for a receptor expressed by the target cells. Either method of targeting can be selected depending on the type of cell or location of tissue to be modified for insulin expression.

Administration of a viral particles or vector encoding a secretable BTC to an individual can be as a single treatment or as multiple treatments depending on the level of BTC secretion desired or on the number of cells to be modified. Methods for the delivery of nucleic acid sequences encoding for a polypeptide are known in the art as described, for example, by Felgner et al., U.S. Pat. No. 5,580,859, issued Dec. 3, 1996. Multiple administrations also can be performed to increase the proportion of modified cells, to increase the number of copies of BTC per cell, or to maintain the effective number of modified cells for a desired duration. Efficacy of the in vivo treatment is achieved if at least one of the symptoms of diabetes is alleviated or reduced. A reduction in severity of a symptom of diabetes in a treated individual can be determined as described previously by one skilled in the art.

It is understood that modifications which do not substantially affect the activity of the various embodiments of this invention are also included within the definition of the invention provided herein. Accordingly, the following examples are intended to illustrate but not limit the present invention.

### EXAMPLE I

### Long-Term Remission of Diabetes by Betacellulin-Induced β Cell Regeneration

This Example shows the treatment of diabetes through betacellulin expression in the pancreases.

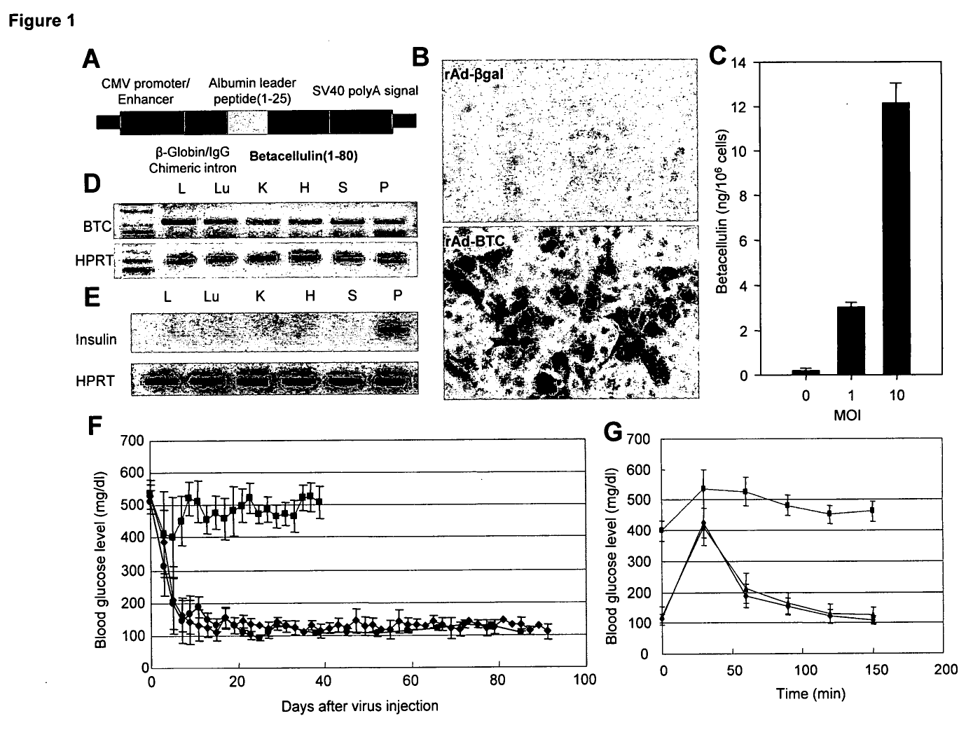

For introduction into a diabetic animal model and in vivo expression of betacellulin (BTC) a recombinant adenoviral vector was constructed. The vector, termed rAd-CMV-BTC, contains 5′ to the BTC coding region the cytomegalovirus (CMV) promoter/enhancer and enhancer region, a β-globin chimeric intron, and an albumin leader sequence, to facilitate secretion of BTC. Located 3′ to the BTC coding region is the SV40 polyadenylation signal sequence. The BTC[1-80] cDNA, encoding mature BTC was inserted between these 5′ and 3′ expression and regulatory regions. A schematic of rAd-CMV-BTC is shown in FIG. 1A. The complete nucleotide and deduced amino acid sequence of BTC[1-80] cDNA encoding mature BTC is set forth as SEQ ID NOS:1 and 2, respectively. The complete nucleotide sequence of rAd-CMV-BTC is set forth as SEQ ID NO:9. The encoded BTC propeptide of SEQ ID NO:9 containing the albumin signal sequence is set forth as SEQ ID NO:10.

Recombinant adenoviruses expressing human BTC cDNA (rAd-BTC) were produced as follows. Briefly, human BTC cDNA encoding the complete 80-amino acid protein was purchased from American Type Culture Collection (ATCC #1887012). The cDNA was cloned into pCR259 (Qbiogene) adenoviral transfer vector at SmalI and NotI sites. The albumin leader peptide sequence was then inserted at SalI and SmaI sites, and the 6-bp sequence, which was additionally inserted by the SmaI recognition sequence, was removed by site-directed mutagenesis. The resultant expression cassette contained a cytomegalovirus (CMV) promoter, β-globin/IgG chimeric intron, and simian virus (SV)40 poly A signal. An adenoviral vector carrying this cassette was constructed using Transpos-Ad™ method (Qbiogene) according to the manufacturer's protocol. The adenoviral vector was linearized with PacI and transfected into HEK-293 cells using lipofectamine-Plus (Invitrogen). Viruses were harvested at 2 weeks after the transfection and used for stock. Viruses were amplified by infecting HEK-293 cells with the stock viruses and purified by CsCl₂-gradient ultracentrifugation as described by Becker et al., Methods Cell. Biol. 43 Pt A, 161 (1994). As a control, recombinant adenoviruses expressing β-galactosidase (rAd-βgal) were produced by replacing BTC cDNA with β-galactosidase cDNA. The viral titer was determined by PCR and the plaque formation unit method using the method described by Prevec, G. L., Biotechnology 20, 363 (1992).

First, the expression and secretion of BTC by the rAd-BTC construct was examined in vitro. An immortalized human hepatocyte line, TTNT-16 cells (Okitsu et al., Diabetes 53, 105 (2004)), was infected with rAd-CMV-BTC, and the production and secretion of BTC were examined by immunohistochemical staining and ELISA, respectively, using anti-BTC antibody (R&D systems, USA).

The results are shown in FIGS. 1B and C. Briefly, FIG. 1B shows the expression of BTC was determined by staining with anti-human BTC antibody at 24 h after infection of 1×10⁴ TTNT-16 cells with either rAd-BTC or rAd-βgal at 5 MOI. The results indicate that BTC was clearly produced in rAd-BTC-infected TTNT-16 cells, but not in cells infected with recombinant adenovirus expressing β-galactosidase (rAd-CMV-βgal). FIG. 1C shows the level of BTC secretion into the supernatant following infection of 1×10⁶ TTNT-16 cells with either rAd-BTC at 1 or 10 MOI and incubated for 24 h. Uninfected TTNT-16 cells were used as a control (0 MOI). The amount of secreted BTC was found to be depended on the dose of rAd-CMV-BTC. Together, these results indicate that cells infected with rAd-BTC efficiently express and secrete BTC.

Second, the expression of BTC by rAd-CMV-BTC was examined in vivo. Briefly, six week-old male nonobese diabetic/severe combined immunodeficiency (NOD.scid) mice (Jackson Labs) were made hyperglycemic by two injections of streptozotocin (STZ; 100 mg/kg body weight, in citrate buffer, pH 4.5, i.p.), and diabetic mice were intravenously injected with rAd-CMV-BTC or rAd-CMV-βgal as a control. STZ-induced diabetic NOD.scid mice or spontaneous autoimmune diabetic NOD mice (blood glucose>500 mg/dl) were injected intravenously with 2×10¹¹ particles for NOD.scid mice or 4×10¹¹ particles for NOD mice of rAd-CMV-BTC or rAd-CMV-βgal, as a control, via the tail vein under methoxyflurane anesthesia. Blood glucose levels were measured every other day. To prevent immune attack of the newly generated β cells in autoimmune diabetic NOD mice, CFA (100 μl/mouse, single i.p. injection) and/or hCG (50 IU/mouse, i.p. daily for 3 weeks) was injected 3 days prior to virus injection. Glucose tolerance tests were performed at 4 weeks after virus injection as previously described by Lee et al., Nature 408, 483 (2000).

Expression of BTC mRNA and insulin in various tissues was examined, including the liver, pancreas, spleen, heart, lung, and kidney, by RT-PCR at 4 weeks after injection. Briefly, various tissues were removed from STZ-induced diabetic NOD.scid mice treated with rAd-BTC and XX weeks after virus injection, and the expression of BTC mRNA and insulin mRNA was analyzed by RT-PCR using the primers: 5′-AGTGGGTAACCTTTATTTCC-3′ (SEQ ID NO:11) and 5′-GTAAAACAAGTCAACTCTCTC-3′ (SEQ ID NO:12) for human BTC and 5′-AGGCTTTTGTCAAGCAG-3′ (SEQ ID NO:13) and 5′-CTGATCTACAATGCCACG-3′ (SEQ ID NO:14) for mouse insulin.

In vivo expression results are shown in FIG. 1D (BTC) and E (insulin) where the legend symbols correspond to: L: liver, Lu: lung, K: kidney, H: heart, S: spleen, P: pancreas. Expression of insulin in these tissues also was examined since islet-like cells have been reported to generate only in the liver of STZ-induced diabetic mice injected with gutless adenovirus expressing NeuroD and BTC (Kojima et al., Nat. Med. 9, 596 (2003)). The expression of HPRT was used as an internal control. BTC mRNA was found to be detected in all tested tissues with the major expression being observed in the liver. These results indicate that BTC is clearly expressed in mice treated with rAd-CMV-BTC. In contrast, that insulin mRNA was detected only in pancreatic tissues of rAd-BTC-treated mice, but not in other tissues including the liver.

Third, whether injection of STZ-induced diabetic NOD.scid mice with rAd-CMV-BTC results in the remission of diabetes also was examined. The results are shown in FIG. 1F where rAd-CMV-BTC (2×10¹¹ particles) was administrated into STZ-induced diabetic NOD.scid (♦) and Balb/c mice (●) and blood glucose levels were measured. As a control, the same dose of rAd-CMV-bgal was administrated into STZ-induced diabetic NOD.scid mice (▪). Blood glucose levels were observed to gradually decrease and reached normal levels within 2 weeks after virus injection. Normoglycemia was maintained for more than 100 days until the end of the study. In contrast, mice treated with rAd-CMV-βgal showed persistent hyperglycemia and died.

Glucose tolerance tests in STZ-induced diabetic mice that achieved normoglycemia after rAd-CMV-BTC treatment also were performed. The results are shown in FIG. 1G where STZ-induced diabetic NOD.scid mice in which blood glucose levels were normalized after rAd-CMV-BTC treatment (▴) were fasted for 4 h and injected with glucose (2 g/kg body weight, i.p.). Blood glucose levels were measured at the indicated times after glucose injection. Untreated NOD.scid mice (♦) and rAd-βgal-treated mice (▪) were used as controls.

The results indicate that rAd-CMV-BTC-treated mice showed the same kinetics of glucose clearance as normal mice. These results contrast with a previous study, in which BTC expression alone had no effect on blood glucose levels in STZ-induced diabetic mice (Kojima et al., Nat. Med. 9:596 (2003)). This difference in the efficacy of BTC gene therapy can be due to differences in the vector and BTC gene construction as well as the mode of expression. For example, this study employed an adenoviral vector, which shows a higher transduction efficiency than the helper-dependent adenoviral vector that was used in the previous study. Further, an albumin leader sequence was inserted in front of the BTC cDNA to facilitate secretion and the cytomegalovirus promoter/enhancer and β-globin chimeric intron for was used for strong expression of the BTC transgene. The results described herein showing the complete remission of diabetes by BTC gene therapy also were confirmed by two different independent investigators in our center.

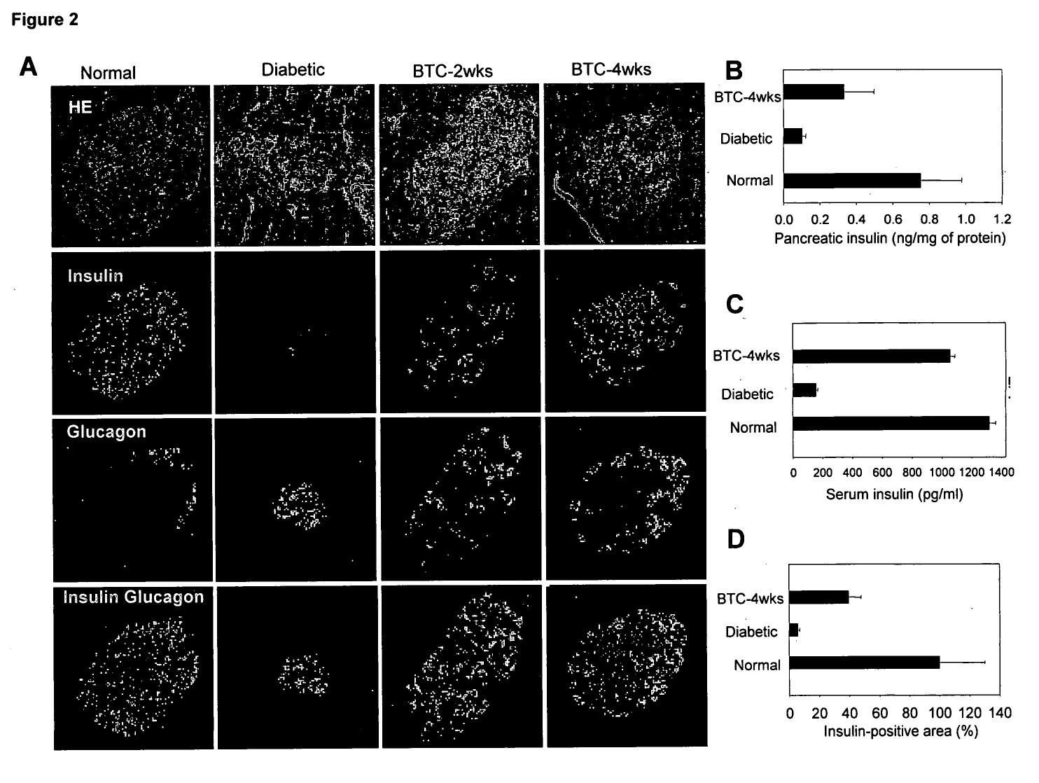

To determine whether treatment of diabetic NOD.scid mice with rAd-CMV-BTC results in the increase of insulin-producing cells, liver and pancreatic sections were stained with anti-insulin antibody and the number of insulin-positive cells was determined. The results are shown in FIGS. 2A and B. Briefly, STZ-induced diabetic NOD.scid mice were injected with rAd-CMV-BTC (2×10¹¹ particles) and sacrificed at 2 weeks (BTC-2 wks) or 4 weeks (BTC-4 wks) later. rAd-βgal-treated diabetic NOD.scid mice (Diabetic) and untreated NOD.scid mice (Normal) were used as controls. Panel A shows where pancreata were removed and sections were stained with hematoxylin and eosin (HE) or anti-insulin antibody (Red) or anti-glucagon antibody (Green). In panel B, the insulin content of the pancreas was measured by radioimmunoassay ELISA.

As shown in FIGS. 2A and B, at 4 weeks after the treatment, the insulin-positive cells were observed to be five-fold higher in the pancreas of rAd-CMV-BTC-treated mice than in the pancreas of rAd-CMV-βgal-treated mice and reached about 40% of those in the pancreas of normal mice. Insulin-positive cells were not observed in the liver. When islets were double-stained with anti-insulin and anti-glucagon antibodies at 2 weeks after rAd-CMV-BTC treatment, insulin-producing cells were found to be interspersed with glucagon-producing a cells in the islets of rAd-CMV-BTC-treated mice, probably due to relocalization of glucagon-producing cells after the destruction of β cells by STZ and subsequent appearance of newly formed β cells. However, β cells were clustered centrally and surrounded by a cells, as found in normal mice, at 4 weeks after rAd-BTC treatment (FIG. 2A), probably due to the continuous increase of newly formed β cells in the central area of the islet.

To confirm that rAd-BTC treatment results in the increase of insulin-producing cells in the pancreas, insulin was extracted from the pancreas or plasma of rAd-BTC- and rAd-βgal-treated mice and normal mice and the concentration was measured by radioimmunoassay as described by Yoon and Notkins, J. Exp. Med. 143:1170 (1976) and Yoon et al., Nature 264:178 (1976). The results are shown in FIGS. 2C and D where panel C shows the serum insulin content measured by ELISA at 30 min after glucose loading (2 g/kg body weight) and prior to sacrifice. Panel D shows measurement of the insulin-positive area after anti-insulin antibody staining and expressed as a percentage of the area found in normal mice. p<0.01 compared with rAd-CMV-βgal-treated diabetic mice.

The show that insulin levels of rAd-CMV-BTC-treated mice (269±31 ng/mg pancreas) were significantly higher than rAd-CMV-βgal-treated mice (116±22 ng/mg pancreas), although lower than normal mice (752±68 ng/mg pancreas) (FIG. 2C). Plasma insulin levels also were determined after glucose loading by ELISA and found that plasma insulin levels in rAd-CMV-BTC-treated mice were significantly increased as compared with rAd-βgal-treated diabetic mice, although lower than in normal mice (FIG. 2D). Taken together, these results show that insulin-producing cells were clearly increased in the pancreas of rAd-CMV-BTC-treated mice, indicating that the remission of diabetes by rAd-BTC treatment was mainly due to the regeneration of β cells in the pancreas. Possible mechanisms involved in the regeneration of cells in the pancreas of rAd-CMV-BTC-treated mice include the replication of pre-existing β cells (Dor et al., Nature 429:41 (2004)), transdifferentiation of non-β cells to β cells (Watada et al., Diabetes 45:1826 (1996); Yoshida et al., Diabetes 51:2505 (2002); Mashima et al., Endocrinology 137:3969 (1996), and Ishiyama et al., Diabetologia 41:623 (1998)), and neogenesis of β cells from adult stem/progenitor cells within the pancreas Trucco, M., J. Clin. Invest. 115:5 (2005); Bouwens, L., Microsc. Res. Tech. 43:332 (1998), and Weir and Bonner-Weir, Nat. Biotechnol. 22:1095 (2004)

BTC has been shown to bind ErbB receptors and induce receptor homo- or hetero-dimerization, autophosphorylation, and subsequent activation of downstream signaling pathways, resulting in cell proliferation and differentiation (Riese et al., Oncogene 12:345 (1996)). The expression of ErbB-1 and ErbB-4 has been found mainly in islets and ductal cells of the normal human pancreas, respectively (Miyagawa et al., Endocr. J. 46:755 (1999)), and ErbB-2, ErbB-3, and ErbB-4 were shown to be expressed in the pancreas during fetal pancreatic development (Kritzik et al., J. Endocrinol. 165:67 (2000)). As well, the expression of ErbB-2 was found to be induced in islet cells adjacent to the areas infiltrated by immunocytes in NOD mice. Several ligands, including BTC, epidermal growth factor, and neuregulins, were shown to mediate the phosphorylation and activation of ErbB-2 through heterodimerzation with ErbB-1, ErbB-3 or ErbB-4 (Kritzik et al., supra). Whether the regeneration of P cells in rAd-CMV-BTC-treated diabetic mice is mediated by ErbB receptors also was determined

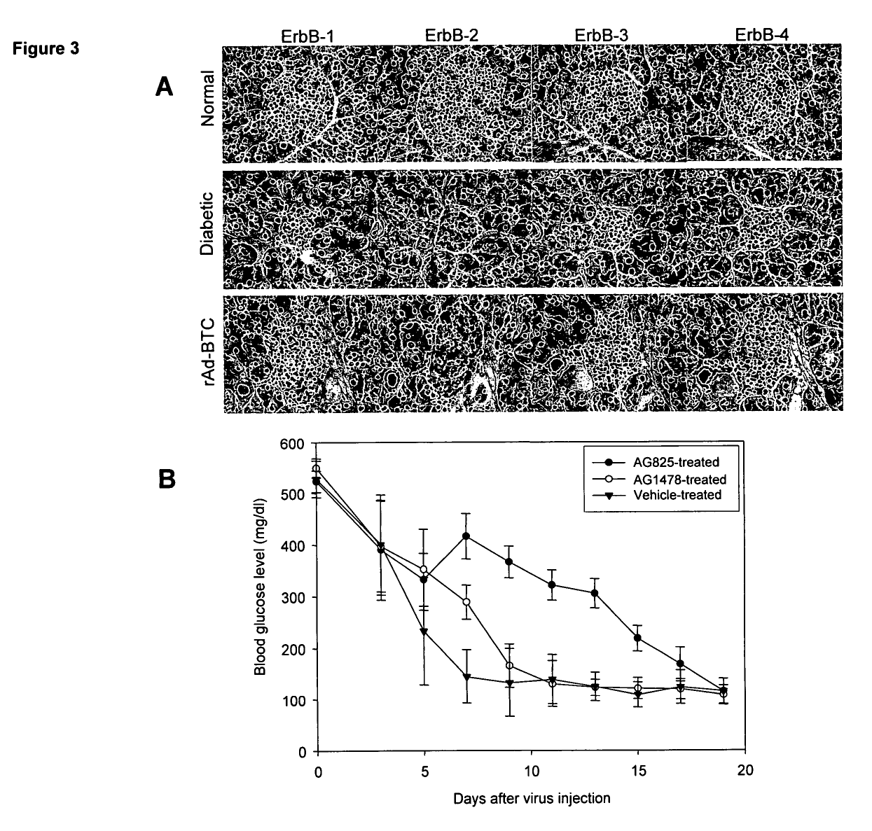

In this regard, the expression of ErbB-1, -2, -3 and -4 in the pancreatic islets of normal mice, STZ-induced diabetic mice, and rAd-CMV-BTC-treated diabetic mice by immunohistochemical staining with anti-ErbB antibodies. The results shown in FIG. 3 demonstrate that ErbB-2 is involved in the remission of diabetes by rAd-CMV-BTC. Briefly, panel A shows pancreatic sections prepared from rAd-CMV-BTC-treated and rAd-CMV-βgal-treated (Diabetic) STZ-induced diabetic mice as well as from untreated (Normal) NOD.scid mice that were stained with anti-ErbB-1, -2, -3, or -4 antibodies. Photomicrographs of representative islets are shown. Panel B shows STZ-induced diabetic NOD.scid mice treated with rAd-CMV-BTC (2×10¹¹ particles). At 3 days after virus injection, mice were treated with vehicle (♥), AG1478 (∘), an ErbB-1 receptor inhibitor, or AG825 (●), an ErbB-2 receptor inhibitor (500 μg in Captisol, i.p.) twice daily for 10 days. Blood glucose levels were measured.

Immunohistochemical analyses were performed by fixing pancreas in methacam (60% methanol v/v, 30% chloroform v/v, and 10% glacial acetic acid v/v) for overnight and processed with two changes of methyl alcohol, two changes of methyl benzoate, xylene and embedded with paraffin. After deparaffinization and rehydration, tissue sections were placed in oven (95° C. for 15 min, 10 mM citrate, pH 6.0) for antigen retrieval and blocked with the blocking solution (5% goat or horse serum, 1% BSA and 0.05% Tween-20 in PBS). Tissues were then incubated with primary antibody solutions; guinea-pig anti-insulin (DAKO, dilution 1:500), rabbit anti-glucagons(DAKO, dilution 1:200), goat anti-ErbB-1 and rabbit anti-ErbB-2,-3, and -4 (Santacruz, dilution 1:100). For secondary antibodies, Cy3-conjugated goat anti-guinea pig IgG (Jackson ImmunoRes., PA dilution 1:200) and Cy2-conjugated anti-rabbit IgG (Jackson ImmunoRes., PA dilution 1:200), HRP-conjugated goat anti-rabbit IgG (Chemicon, dilution 1:500), and HRP-conjugated horse anti-goat IgG (Chemicon, dilution 1:500) were used. Fluorescence was imaged using laser scanning confocal fluorescent microscope (Zeiss LSM 510) and peroxidase staining was performed with VIP as a chromogen (violet color) (VIP kit; Vector Laboratories).

In vivo treatment with tyrosine kinase inhibitors was performed by injecting STZ-induced diabetic NOD.scid mice with rAd-CMV-BTC (2×10¹¹ particles, i.v.) and the tyrosine kinase inhibitors, AG1478 or AG825 (500 μg) in 100 μl of 100 mM Captisol (Cydex Inc.) was intraperitoneally injected twice daily for 10 days beginning on the third day after virus injection. Vehicle alone (100 mM Captisol) was injected as a control. The statistical significance of the differences between groups for all studies was analyzed by Student's t test. A level of P<0.05 was accepted as significant.

ErbB-1 was observed to be weakly expressed in islets from all of these mice, and ErbB-3 and ErbB-4 expression was not measurable in islets from any of these mice. In contrast, ErbB-2 was highly expressed in pancreatic islets of both rAd-CMV-BTC-treated and untreated STZ-induced diabetic mice as compared with normal mice (FIG. 3A). Because ErbB-1 and ErbB-2 receptors were found to be expressed in the islets, their involvement in BTC-induced remission of diabetes was assessed. Blood glucose levels were measured following injection of AG1478 or AG825, specific blockers of ErbB-1 and ErbB-2 receptor signaling pathways, respectively (Levitzki and Gazit, Science 267:1782 (1995)), for 10 days into STZ-induced diabetic, rAd-CMV-BTC-treated NOD.scid mice beginning on the third day after virus injection. Injection of A825 was found to abrogated the remission of diabetes by rAd-BTC. However, when AG825 injections were stopped on the thirteenth day after virus injection, blood glucose levels gradually decreased and became normoglycemic (about 100 mg/dl) at 23 days after virus injection. The effect of AG1478 on the abrogation of BTC-induced remission of diabetes was less pronounced than that of AG825 (FIG. 3B). It is unclear whether this difference is due to incomplete blocking of the ErbB-1 receptor by AG1478 or functional differences in the interaction of BTC with ErbB-1 and ErbB-2. Nevertheless, these results indicate that ErbB-2 is involved in the remission of diabetes by BTC.

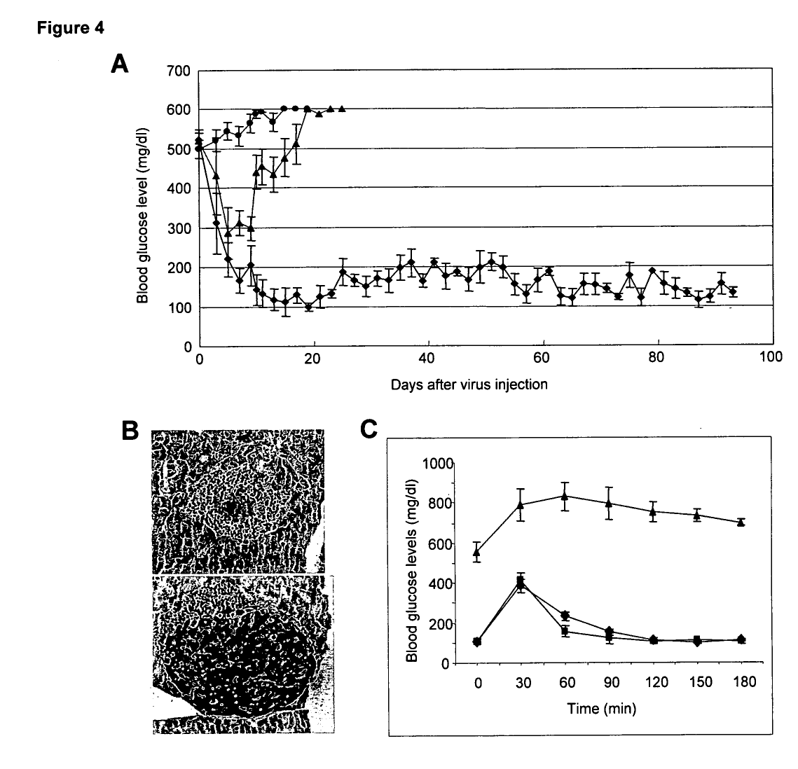

The efficacy of rAd-CMV-BTC gene therapy in autoimmune diabetic NOD mice, a model of human autoimmune type 1 diabetes was examined. Injection of rAd-CMV-BTC (4×10¹¹ particles, i.v.) into newly developed diabetic NOD mice (blood glucose levels>500 mg/dl) was performed and the changes in blood glucose levels was assessed. The results are shown in FIG. 4 where panel A shows autoimmune diabetic NOD mice (blood glucose levels>500 mg/dl) injected with CFA (100 ul) subcutaneously for 3 days prior to injection of rAd-BTC (2×10¹¹ particles). ♦, rAd-BTC with CFA; ▴, rAd-BTC only; ●, CFA only. Panel B shows an anti-insulin antibody staining of the pancreas of rAd-CMV-BTC/CFA treated NOD mice at 3 months after virus injection. Several islets were strongly stained with anti-insulin antibody and surrounded with T cells but significant infiltration was not found. The top portion of the panel corresponds to islets from untreated diabetic NOD whereas the bottom portion corresponds to islets from rAd-CMV-BTC/CFA-treated NOD. Panel C illustrates the results of a glucose tolerance test. Diabetic NOD mice in which blood glucose levels were normalized after rAd-BTC/CFA treatment (♦) were fasted for 4 h and injected with glucose (2 g/kg body weight, i.p.). Blood glucose levels were normalized at the indicated times after glucose injection. CFA only-treated diabetic NOD (▴) and nondiabetic NOD mice (▪) were used as controls.

Blood glucose levels were observed to decrease to below 300 mg/dl at 4-5 days after virus injection, but returned to levels above 500 mg/dl at 2 weeks after virus injection, probably due to re-attack of regenerated insulin-producing cells by autoimmune responses (FIG. 4A). Complete Freund's adjuvant (CFA) has been shown to prevent diabetes in NOD mice, possibly by the induction of immunoregulatory T cells (Qin et al., J. Immunol. 150:2072 (1993)). As well, a recent study showed that the control of the finely tuned immune balance between effector and regulatory T cells results in the prevention of autoimmune diabetes in NOD mice (Khil et al. Diabetes 53 (Suppl. 1), A43 (2004)). Therefore, control of the immune balance in diabetic NOD mice was assessed by injection of CFA before treatment with rAd-CMV-BTC. Blood glucose levels were examined following injection of CFA (100 μl, i.p.) into newly developed diabetic NOD mice and treatment with rAd-CMV-BTC (4×10¹¹ particles) 3 days later. The results showed that blood glucose levels were normalized at 2-3 weeks after virus injection, and normoglycemia was maintained for over 90 days until the end of the experiment. Treatment of diabetic NOD mice with CFA alone had no effect on blood glucose levels (FIG. 4A).

When pancreatic sections of rAd-CMV-BTC-treated NOD mice at 3 months after virus injection was examined, regenerated islets were found surrounded by immunocytes, but were not infiltrated by them (FIG. 4B). There was no significant difference in the clearance of blood glucose between rAd-CMV-BTC-treated NOD mice injected with CFA and normal mice was found in glucose tolerance tests in these mice. In contrast, blood glucose levels of rAd-CMV-βgal-treated NOD control mice injected with CFA were significantly higher than rAd-BTC-treated mice at all times measured (FIG. 4C). These results indicate that rAd-BTC gene therapy can result in the complete remission of autoimmune diabetes when the immune balance is properly controlled.

Using two animal models of diabetes, chemically induced and spontaneous autoimmune diabetic mice, the results above show that the constitutive expression and secretion of BTC in rAd-CMV-BTC-treated mice induced the regeneration of insulin-producing cells in the pancreas through ErbB-2 receptors, resulting in long-term, complete remission of diabetes. The success of this BTC gene therapy in animal models demonstrates therapeutic usefulness for the cure of type 1 diabetes in humans, along with immunological strategies to halt the autoimmune attack of regenerated β cells. BTC gene therapy would overcome the shortage of immunologically matched donor islets that is limiting transplantation therapy and does not require any surgical procedures.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A vector comprising a nucleic acid operably linking a promoter, an intron, a secretory leader sequence encoding nucleic acid, a human betacellulin (BTC) encoding nucleic acid, or functional fragment thereof, and a polyadenylation signal sequence, wherein expression of BTC produces a secreted, mature BTC.

2. The vector of claim 1, wherein said vector comprises an adenoviral vector.

3. The vector of claim 1, wherein said secretory leader sequence encoding nucleic acid is an albumin or immunoglobulin kappa chain leader sequence.

4. The vector of claim 1, wherein said secretory leader sequence encoding nucleic acid comprises a nucleotide sequence encoding an albumin secretory leader sequence.

5. The vector of claim 1, wherein said human BTC encoding nucleic acid comprises substantially the same nucleotide sequence as shown as SEQ ID NO:1.

6. The vector of claim 1, wherein said human BTC encoding nucleic acid comprises a nucleotide sequence encoding substantially the same amino acid sequence as that shown as SEQ ID NO:2.

7. The vector of claim 4, wherein said albumin secretory leader sequence encoding nucleic acid comprises substantially the same nucleotide sequence as SEQ ID NO:7.

8. The vector of claim 4, wherein said albumin secretory leader sequence encoding nucleic acid comprises a nucleotide sequence encoding substantially the same amino acid sequence as nucleotides 1-72 of SEQ ID NO:10.

9. A vector comprising a nucleic acid operably linking a cytomegalovirus (CMV) promoter and enhancer region, a β-globin chimeric interon, an albumin leader sequence encoding nucleic acid, a human betacellulin (BTC) encoding nucleic acid, or a functional fragment thereof, and an SV40 polyadenylation signal sequence, wherein expression of BTC produces a secreted, mature BTC.

10. The vector of claim 9, wherein said vector comprises an adenoviral vector.

11. The vector of claim 9, wherein said human BTC encoding nucleic acid comprises substantially the same nucleotide sequence as shown as SEQ ID NO:1.

12. The vector of claim 9, wherein said human BTC encoding nucleic acid comprises a nucleotide sequence encoding substantially the same amino acid sequence as that shown as SEQ ID NO:2.

13. The vector of claim 9, wherein said albumin secretory leader sequence encoding nucleic acid comprises substantially the same nucleotide sequence as SEQ ID NO:7.

14. The vector of claim 9, wherein said albumin secretory leader sequence encoding nucleic acid comprises a nucleotide sequence encoding substantially the same amino acid sequence as nucleotides 1-72 of SEQ ID NO:10.

15. The vector of claim 9, comprising the nucleotide sequence as shown as SEQ ID NO:9.

16. A host cell containing the vector of claims 1, 9 or 15.

17. A method of treating or preventing diabetes, comprising administering to an individual an effective amount of a viral particle having a vector expressing a secreted, mature human betaculin (BTC) or a functional fragment thereof, said vector comprising a nucleic acid operably linking a promoter, an intron, a secretory leader sequence encoding nucleic acid, a human betacellulin (BTC) encoding nucleic acid, or functional fragment thereof, and a polyadenylation signal sequence.

18. The method of claim 17, wherein said vector comprises an adenoviral vector.

19. The method of claim 17, wherein said secretory leader sequence encoding nucleic acid is an albumin or immunoglobulin kappa chain leader sequence.

20. The method of claim 17, wherein said secretory leader sequence encoding nucleic acid comprises a nucleotide sequence encoding an albumin secretory leader sequence.

21. The method of claim 17, wherein said human BTC encoding nucleic acid comprises substantially the same nucleotide sequence as shown as SEQ ID NO:1.

22. The method of claim 17, wherein said human BTC encoding nucleic acid comprises a nucleotide sequence encoding substantially the same amino acid sequence as that shown as SEQ ID NO:2.

23. The method of claim 20, wherein said albumin secretory leader sequence encoding nucleic acid comprises substantially the same nucleotide sequence as SEQ ID NO:7.

24. The method of claim 20, wherein said albumin secretory leader sequence encoding nucleic acid comprises a nucleotide sequence encoding substantially the same amino acid sequence as nucleotides 1-72 of SEQ ID NO:10.

25. The method of claim 17, wherein said vector is administered in a pharmaceutically acceptable carrier.


SEQUENCE LISTING (14 sequences)

SEQ ID NO: 1 - DNA, 240 bp, Homo sapiens
  CDS (1)...(240)
gatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttac

SEQ ID NO: 2 - protein, 80 aa, Homo sapiens
DGNSTRSPETNGLLCGDPEENCAATTTQSKRKGHFSRCPKQYKHYCIKGRCRFVVAEQTPSCVCDEGYIGARCERVDLFY

SEQ ID NO: 3 - protein, 178 aa, Homo sapiens
MDRAARCSGASSLPLLLAIALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 4 - protein, 178 aa, Bos taurus
MARAAPGSGASPLPLLPALALGLVILHCVVADGNSTRSPEDDGLLCGDHAENCPATTTQPKRRGHFSRCPKQYKHYCIKGRCRFVVAEQTPSCVCDEGYAGARCERVDLFYLRGDRGQILVICLIAVMVIFIILVVSICTCCHPLRKRRKRRKKEEEMETLGKDITPINDDIQETSIA

SEQ ID NO: 5 - protein, 177 aa, Mus musculus
MDPTAPGSSVSSLPLLLVIALGLAILHCVVADGNTTRTPETNGSLCGAPGENCTGTTPRQKVKTHFSRCPKQYKHYCIHGRCRFVVDEQTPSCICEKGYFGARCERVDLFYLQQDRGQILVVCLIVVMVVFIILVTGVCTCCHPLRKHRKKKKEEKMETLDKDKTPISEDIQETNIA

SEQ ID NO: 6 - protein, 128 aa, Artificial Sequence
  Consensus alignment sequence of BTC
MAPSSLPLLLALGLILHCVVADGNTRPEGLCGENCTTKHFSRCPKQYKHYCIGRCRFVVEQTPSCCGYGARCERVDLFYLDRGQILVCLIVMVFIILVCTCCHPLRKRKKKEEMETLKDTPIDIETIA

SEQ ID NO: 7 - DNA, 75 bp, Artificial Sequence
  exemplary hypothetical leader sequence - not derived from any organism
atgaagtgggtaacctttatttcccttctttttctctttagctcggcttattccaggggtgtgtttcgtcgagat

SEQ ID NO: 8 - DNA, 63 bp, Artificial Sequence
  exemplary hypothetic leader sequence - not derived from any organism
atggagacagacacactcctgctatgggtactgctgctctgggttccaggttccactggtgac

SEQ ID NO: 9 - DNA, 1647 bp, Artificial Sequence
  synthetic adenoviral vector sequence
  promoter (1)...(795): CMV Promoter/Enhancer
  intron (857)...(989): B-Globin/IgG Chimneric Intron
  polyA_signal (1426)...(1647): SV40 late PolyASignal
  sig_peptide (1095)...(1166): Albumin leader sequence
  CDS (1167)...(1406): Betacellilin[1-80] cDNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccatgaagtgggtaacctttatttcccttctttttctctttagctcggcttattccaggggtgtgtttcgtcgagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttactaggggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 10 - DNA, 312 bp, Homo sapiens
atgaagtgggtaacctttatttcccttctttttctctttagctcggcttattccaggggtgtgtttcgtcgagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttac

SEQ ID NO: 11 - DNA, 20 bp, Artificial Sequence
  PCR primer
agtgggtaacctttatttcc

SEQ ID NO: 12 - DNA, 21 bp, Artificial Sequence
  PCR primer
gtaaaacaagtcaactctctc

SEQ ID NO: 13 - DNA, 17 bp, Artificial Sequence
  PCR primer
aggcttttgtcaagcag

SEQ ID NO: 14 - DNA, 18 bp, Artificial Sequence
  PCR primer
ctgatctacaatgccacg